(19) United States
(12) Patent Application Publication
(10) Pub. No.: US 2015/0140588 A1
(43) Pub. Date: May 21, 2015

(54) Lipase Variants and Polynucleotides Encoding Same

(71) Applicants: Werner Besenmatter, Wiener Neudorf (AT); Allan Svendsen, Hoersholm (DK)

(72) Inventors: Werner Besenmatter, Wiener Neudorf (AT); Allan Svendsen, Hoersholm (DK)

(73) Assignee: Novozymes A/S, Bagsvaerd (DK)

(21) Appl. No.: 14/124,929

(22) PCT Filed: Jul. 3, 2012
(86) PCT No.: PCT/EP2012/062896

§ 371 Date: Jun. 16, 2014

Related U.S. Application Data
(60) Provisional application No. 61/543,964, filed on Oct. 6, 2011.
(60) Provisional application No. 61/508,787, filed on Jul. 18, 2011.

(30) Foreign Application Priority Data
Jul. 15, 2011 (EP) 11174169.0
Oct. 6, 2011 (EP) 1184110.2

(51) Int. Cl. C12N 9/20

(52) U.S. Cl. CPC C12N 9/20; C12Y 301/01003
USPC 435/19; 435/198; 536/23.2; 435/320.1; 506/11; 435/252.3; 435/252.31; 435/252.35; 435/252.33; 435/252.34; 435/325; 435/348; 435/419; 435/254.11; 435/254.2; 435/254.23; 435/254.22; 435/254.21; 435/254.3; 435/254.7; 435/254.8; 435/254.4; 435/254.5; 435/254.6

(57) ABSTRACT

The present invention relates to variants with improved activity in an amide-bond reaction. The present invention also relates to polynucleotides encoding the variants; nucleic acid constructs, vectors, and host cells comprising the polynucleotides; and methods of using the variants.

### REFERENCE TO A SEQUENCE LISTING

This application contains a Sequence Listing in computer readable form, which is incorporated herein by reference.

### BACKGROUND OF THE INVENTION

1. Field of the Invention

The present invention relates to lipases variants with improved activity in an amide-bond reaction, polynucleotides encoding the variants, methods of producing the variants, and methods of using the variants. More particularly, the present invention relates to modified enzymes with at least 60% homology with Candida antarctica Lipase B (CALB) which have improved activity in an amide-bond reaction as compared to the corresponding unmodified enzyme.

2. Description of the Related Art

Lipases (triacylglycerol hydrolases, EC 3.1.1.3) are serine hydrolases that catalyses the hydrolysis of fatty acid esters (triglycerides). They have broad substrate specificity and accommodate a wide range of structurally diverse esters, alcohols and carboxylic acids as substrates. Lipases do not catalyze or are very poor catalysts for the hydrolysis of amides which in part is explained by the lower reactivity of amides compared to esters (Bruice, P. Y. (1998) Organic Chemistry, 678). However, a few reports of amide hydrolysis by lipases have been published: Duarte D E., Castillo E., Bárzana E., & López-Munguïa A. (2000) Biotechnol Lett., 22, 1811-1814 describes the hydrolysis of capsaicin, a natural water-insoluble amide, by lipase B from Candida antarctica. Henke E. & Bornscheuer U. T. (2003) Anal Chem., 75, 255-260 describes a fluorophoric assay for the high-throughput determination of amide hydrolysis, which was used to evaluate 22 unmodified lipases and esterases, and to screen 15000 mutants created by error-prone PCR and other random mutagenesis methods. This directed evolution experiment revealed no positive result, that is, new amidase activity.

In another directed evolution experiment Fujii R., Nakagawa Y., Hiratake J., Sogabe A., & Sakata K. (2005) Protein Eng Des Sel., 18, 93-101 screened 20000 mutants, which were generated randomly by error-prone PCR, of Pseudomonas aeruginosa lipase for improved amide hydrolysis and found 3 mutations, namely F207S, A213D and F265L to affected the amidase/esterase activity ratios. The continuation of this work was described by Nakagawa Y., Hasegawa A., Hiratake J. & Sakata K. (2007) Protein Eng Des Sel., 20, 339-346, where they mutated Pseudomonas aeruginosa lipase for enhanced amidase activity and showed that the triple mutant F207S+A213D +M252F, but not substitutions of residues M16 or H83, gave an increase in amidase activity. This study also tried to give more insight why lipases do not hydrolyze amides despite some similarity to serine proteases, which are able to hydrolyze amides. Both contain a catalytic triade, which consists of Ser-His-Asp/Glu, and an oxyanion hole. However, there are also considerable differences which are manifested in less than 30% sequence identity between serine proteases and Pseudomonas aeruginosa lipase. Pseudomonas aeruginosa lipase and Candida antarctica lipase B share less than 30% sequence identity. Candida antarctica lipase B is already an established tool in organic chemistry, in particular for ester-bond reactions, that is the hydrolysis and synthesis of esters and transesterifications.

Thus, it would be desirable to generate lipases which are also useful for catalyzing the cleavage (hydrolysis) and formation of amides. The present invention provides lipase variants with improved activity in an amide-bond reaction as compared to the parent lipase.

### SUMMARY OF THE INVENTION

The present invention relates to a variant of a parent lipase which variant is: (a) a polypeptide having at least 60% sequence identity to the mature polypeptide of SEQ ID NO: 2; (b) a polypeptide encoded by a polynucleotide that hybridizes under low stringency conditions with (i) the mature polypeptide coding sequence of SEQ ID NO: 1, or (ii) the full-length complement of (i); (c) a polypeptide encoded by a polynucleotide having at least 60% identity to the mature polypeptide coding sequence of SEQ ID NO: 1; or (d) a fragment of the mature polypeptide of SEQ ID NO: 2, wherein said variant has improved activity in an amide-bond reaction in comparison with the parent lipase.

The present invention relates to a variant comprising a substitution at one or more positions corresponding to positions S31R/K; G39; A225; T103V/S/A; L278G/K/R/H; W104Y/K/R; T42G/S/Q/H/I/L/M; D223G/N/A/V/H; I189; Q191; D134V/T/I/F/A/S/K/R; E188; V221A/R/K/H; 7 P38H/N/A/G; G41P/S; A132; N79; Q106; and/or L140R/K/H of the mature polypeptide of SEQ ID NO: 2.

The present invention also relates to isolated polynucleotides encoding the variants; nucleic acid constructs, vectors, and host cells comprising the polynucleotides; and methods of producing the variants.

The present invention also relates to methods of using of the lipase variants for catalyzing amide-bond reactions.

### BRIEF DESCRIPTION OF THE FIGURES

FIG. 1 shows an alignment of lipase amino acid sequences.

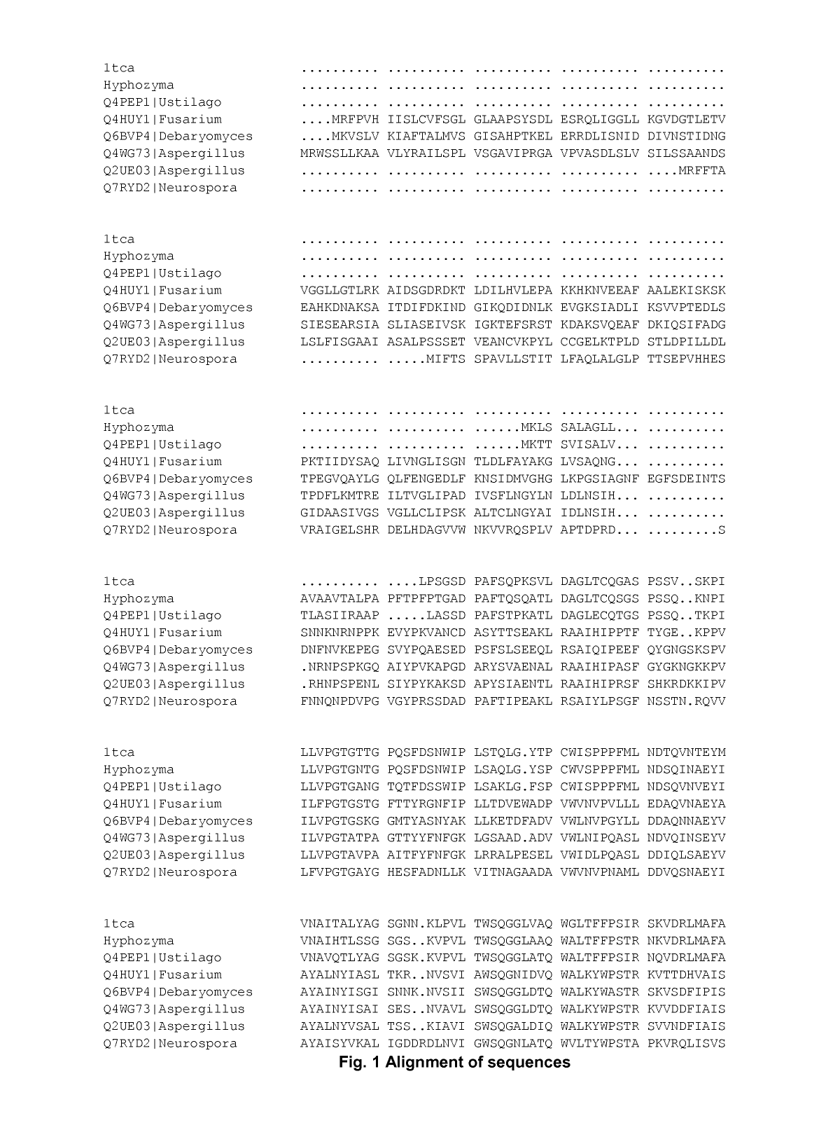

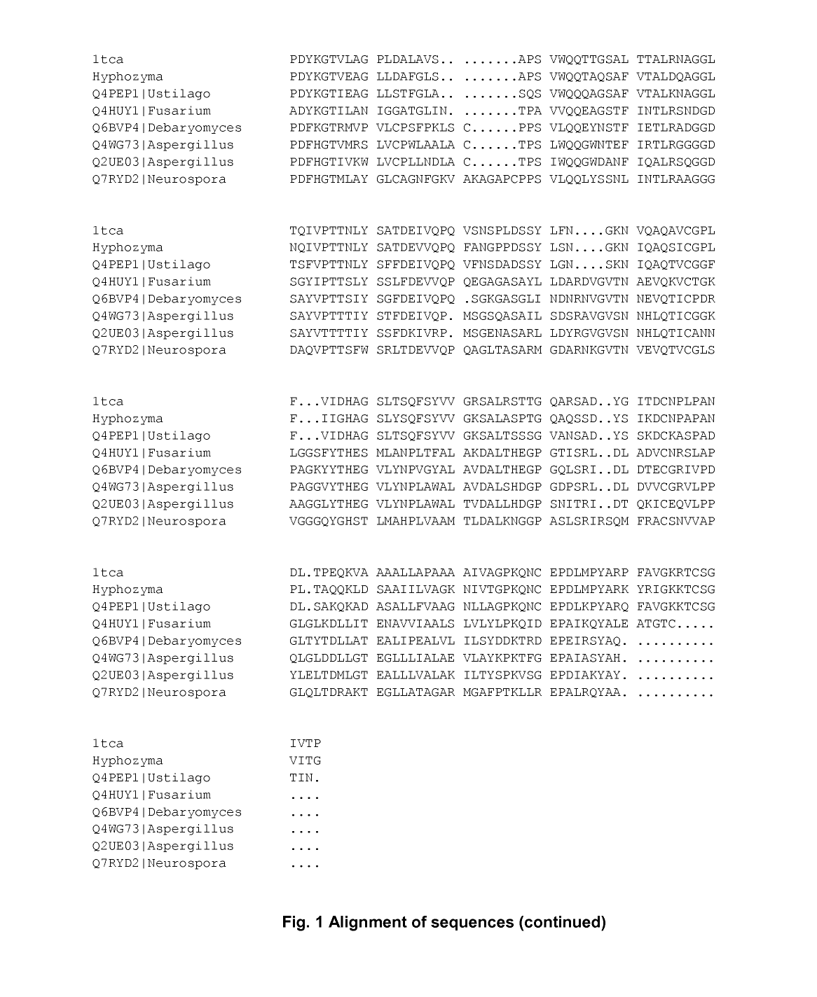

### DEFINITIONS

Activity in an amide-bond reaction: The term “activity in an amide-bond reaction” means formation or cleavage (permanent or transient) of amide bonds, including, but not limited to, amide synthesis, amide hydrolysis, alcoholysis, aminolysis, transamidification, acidolysis, transacylation, acyl transfers and the reversal of these reaction. For purposes of the present invention, activity in an amide-bond reaction is determined according to the procedure described in the Examples. In one aspect, the variants of the present invention have an improved activity in an amide-bond reaction as compared to that of the parent lipase e.g. the mature polypeptide of SEQ ID NO: 2.

Amidase: The term “Amidase” means an enzyme that catalyzes the hydrolysis of an amide.

Esterase: The term “Esterase” means an enzyme that catalyzes the hydrolysis of an ester in the presence of water into an acid and an alcohol.

Lipase: The term “Lipase” means an enzyme that catalyzes the hydrolysis or formation of lipids. For the purpose of this invention the term “Lipase” also includes any enzyme which irrespective of enzyme classification has at least 60% homology with the amino acid residues 1 to 317 of SEQ ID No: 2.

Active site residues: The term “active site residues” means any residue (or backbone part) that gets into direct contact with the substrate, transition state or product.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Fragment: The term “fragment” means a polypeptide having one or more (e.g., several) amino acids absent from the amino and/or carboxyl terminus of a mature polypeptide; wherein the fragment has improved activity in an amide-bond reaction.

High stringency conditions: The term “high stringency conditions” means for probes of at least 100 nucleotides in length, pre-hybridization and hybridization at 42° C. in 5× SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2× SSC, 0.2% SDS at 65° C.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent. Such improved properties relate to increased activity in an amide-bond reaction and include, but are not limited to: catalytic efficiency, catalytic rate, turnover number, specific activity, substrate binding, substrate cleavage, substrate specificity and product release.

Isolated: The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Low stringency conditions: The term “low stringency conditions” means for probes of at least 100 nucleotides in length, pre-hybridization and hybridization at 42° C. in 5× SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2× SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 317 of SEQ ID NO: 2. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having improved activity in an amide-bond reaction. In one aspect, the mature polypeptide coding sequence is nucleotides 76 to 1026 of SEQ ID NO: 1.

Medium stringency conditions: The term “medium stringency conditions” means for probes of at least 100 nucleotides in length, pre-hybridization and hybridization at 42° C. in 5× SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2× SSC, 0.2% SDS at 55° C.

Medium-high stringency conditions: The term “medium-high stringency conditions” means for probes of at least 100 nucleotides in length, pre-hybridization and hybridization at 42° C. in 5×SSPE, 0.3% SDS, 200 micrograms/ml sheared and denatured salmon sperm DNA, and either 35% formamide, following standard Southern blotting procedures for 12 to 24 hours. The carrier material is finally washed three times each for 15 minutes using 2× SSC, 0.2% SDS at 60° C.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration which is a control sequence placed at an appropriate position relative to the coding sequence of a polynucleotide such that the control sequence directs expression of the coding sequence.

Parent or parent lipase: The term “parent” or “parent lipase” means a lipase to which a substitution is made to produce the lipase variants of the present invention. The parent may be a naturally occurring (wild-type) polypeptide or a variant or a fragment thereof.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment with improved activity in an amide-bond reaction.

Variant: The term “variant” means a polypeptide with improved activity in an amide-bond reaction comprising a substitution, at one or more (e.g., several) positions. A substitution means replacement of the amino acid occupying a position with a different amino acid. The variants of the present invention have an improved activity in an amide-bond reaction in comparison with that of the parent lipase.

Very high stringency conditions: The term “very high stringency conditions” means for probes of at least 100 nucleotides in length, pre-hybridization and hybridization at 42° C. in 5× SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2× SSC, 0.2% SDS at 70° C.

Very low stringency conditions: The term “very low stringency conditions” means for probes of at least 100 nucleotides in length, pre-hybridization and hybridization at 42° C. in 5× SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2× SSC, 0.2% SDS at 45° C.

Wild-type: The term “wild-type” means a polypeptide expressed by a naturally occurring organism, such as a bacterium, archaea, yeast, fungus, plant or animal found in nature.

Conventions for Designation of Variants

For purposes of the present invention, the mature polypeptide disclosed in SEQ ID NO: 2 is used to determine the corresponding amino acid residue in another lipase. The amino acid sequence of another lipase is aligned with the mature polypeptide disclosed in SEQ ID NO: 2, and based on the alignment, the amino acid position number corresponding to any amino acid residue in the mature polypeptide disclosed in SEQ ID NO: 2 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another lipase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537:_{—}39-64; Katoh and Toh, 2010, Bioinformatics 26:_{—}1899-1900), and EMBOSS EMMA employing ClustalW (1.83 or later; Thompson et al., 1994, Nucleic Acids Research 22: 4673-4680), using their respective default parameters.

When the other enzyme has diverged from the mature polypeptide of SEQ ID NO: 2 such that traditional sequence-based comparison fails to detect their relationship (Lindahl and Elofsson, 2000, J. Mol. Biol. 295: 613-615), other pair wise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP super families of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homolog (e.g., Holm and Park, 2000, Bioinformatics 16: 566-567).

In describing the variants of the present invention, the nomenclature described below is adapted for ease of reference. The accepted IUPAC single letter or three letter amino acid abbreviations are employed.

Substitutions.

For an amino acid substitution, the following nomenclature is used: Original amino acid, position, substituted amino acid. Accordingly, the substitution of threonine at position 226 with alanine is designated as “Thr226Ala” or “T226A”.

Multiple Substitutions.

Variants comprising multiple alterations are separated by addition marks (“+”), e.g., “Arg170Tyr +Gly195Glu” or “R170Y +G195E” representing a substitution of arginine and glycine at positions 170 and 195 with tyrosine and glutamic acid, respectively.

Different Substitutions.

Where different alterations can be introduced at a position, the different alterations are separated by a comma, e.g., “Arg170Tyr,Glu” or a slash e.g., “R170Y/E” and represents a substitution of arginine at position 170 with tyrosine or glutamic acid. Thus, “Tyr167Gly,Ala +Arg170Gly,Ala” or “Y167G/A +R170G/A” designates the following variants: “Tyr167Gly +Arg170Gly”, “Tyr167Gly +Arg170Ala”, “Tyr167Ala +Arg170Gly”, and “Tyr167Ala +Arg170Ala” or “Y167G +R170G”, “Y167G +R170A”, “Y167A +R170G” and “Y167A +R170A” respectively.

### DETAILED DESCRIPTION OF THE INVENTION

Improved Activity in an Amide-Bond Reaction

Compared to a parent lipase the invention relates to an improvement in the activity of the variant on at least one selected amide bond in at least one substrate. Thus, an enzyme with improved activity in an amide-bond reaction may be useful for many purposes; e.g. in biocatalysis, organic synthesis and analysis such as e.g. in pharmaceutical and pesticide synthesis, analysis and degradation, where amines and amides are key intermediates and products, hydrolysis (e.g. removal of protection groups, degradation of remains), alcoholysis, aminolysis, acidolysis, amide formation, transamidification, racemic resolution of amides, amines, carboxylic acids, esters and alcohols, degradation of unwanted amides, e.g. decontamination of soil, surfaces, food and feed; removal of naturally occurring amides from intermediate goods, modification of products containing amides. It may be desired to increase the activity in an amide-bond reaction for any industrial use where such biocatalysts are used.

Substrates and Products

Substrates or products may be any compound that comprises an amide-bond. Examples include, but are not limited to N-propyl-butyramide, 2-chloro-N-ethyl-acetamide, 2-bromo-N-decyl-acetamide, N-[3-(dimethylamino)-propyl]-lauramide, p-nitrophenyl-amides e.g. p-nitrophenyl-acetamide (=p-nitroacetanilide) p-nitrophenyl-butyramide, N-(2-naphthyl)-amides e.g. N-(2-naphthyl)-oleamide, 4-nitro-N-propyl-benzamide. Further examples are compounds of the following structure:

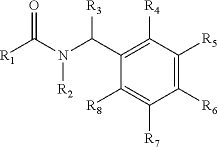

wherein any of the R-groups may be hydrogen (H) or another residue known to occur in organic compounds.

These substrate examples include, but are not limited to N-benzyl-chloroacetamide (=2-chloro-N-benzyl-acetamide), N-benzyl-butyramide, N-benzyl-decanamide, N-benzyl-benzamide, 2,2,2-trifluoro-N-[α-methyl-benzyl]-acetamide, 2,2-dichloro-N-(1-phenylethyl)-acetamide, N-(1-phenylethyl)-octanamide, N-(1-phenylethyl)-2-methoxyacetamide, N-[1-(4-methylphenyl)-ethyl]-octanamide, N-[1-(4-chlorophenyl)-ethyl]-2-methoxyacetamide, N-[1-(4-phenoxyphenyl)-ethyl]-octanamide, N-[1-(naphthalen-2-yl)-ethyl]-acetamide, N-[1-{bicyclo[2.2.1]heptan-1-yl}-ethyl)-octanamide, capsaicin (=8-methyl-N-vanillyl-trans-6-nonenamide), beflubutamid (=(RS)-N-benzyl-2-(α,α,α,4-tetrafluoro-m-tolyloxy)-butyramide), benzipram (=N-benzyl-N-isopropyl-3,5-dimethylbenzamide), bromobutide (=(RS)-2-bromo-3,3-dimethyl-N-(1-methyl-1-phenylethyl)-butyramide), tebutam (=N-benzyl-N-isopropyl-2,2-dimethylpropionamide), ε-aminocaproyl-p-chlorobenzylamide, N-acetyl-procainamide, 3-(4-hydroxyphenyl)-N-benzyl-propionamide, N,N′-dibenzyl-phthalamide.

Alteration Near the Catalytic Triad

In the active site of lipases are three amino acids: a Serine (S), a Histidine (H) and an Aspartic acid (D)/Glutamic acid (E) which forms a catalytic triad. The catalytic triade of SEQ ID No: 2 constitute the residues S105, D187 and H224.

The amino acid sequence of the parent lipase may be amended at at least one amino acid residue located within a distance of 10 Å, 9 Å, 8 Å, 7 Å or 6 Å of any of the residues of the catalytic triad which changes the geometry of the pocket that contains the catalytic triade, affects the catalytic triade through the electrostatic field, or both.

Such amino acid residues that lie within 10 Å of at least one of the catalytic triade residues in SEQ ID No: 2 are: G39; A225; T103; L278; W104; T42; D223; I189; Q191; D134; E188; V221; P38; G41; A132; N79; Q106; and L140. Residues that lie within 10 Å of S105 in SEQ ID No: 2 are: G39; A225; T103; L278; W104; T42; D223; I189; Q191; D134; E188; P38; G41; A132; N79; and Q106. Residues that lie within 10 Å of D187 in SEQ ID No: 2 are: A225; L278; W104; D223; I189; Q191; D134; E188; V221; A132; Q106; and L140. Residues that lie within 10 Å of H224 in SEQ ID No: 2 are: G39; A225; T103; L278; W104; T42; D223; I189; Q191; D134; E188; V221; P38; G41; A132; Q106; and L140.

Such amino acid residues that lie within 8 Å of at least one of the catalytic triade residues in SEQ ID No: 2 are: G39; A225; T103; L278; W104; T42; D223; I189; Q191; D134; E188; V221; P38; G41; A132; Q106; and L140. Residues that lie within 8 Å of S105 in SEQ ID No: 2 are: G39; A225; T103; W104; T42; I189; D134; P38; G41; A132; and Q106. Residues that lie within 8 Å of D187 in SEQ ID No: 2 are: A225; W104; D223; I189; Q191; D134; E188; V221; A132; and L140. Residues that lie within 8 Å of H224 in SEQ ID No: 2 are: G39; A225; L278; W104; D223; I189; Q191; D134; E188; A132; and Q106.

Such amino acid residues that lie within 6 Å of at least one of the catalytic triade residues in SEQ ID No: 2 are: G39; A225; T103; L278; W104; D223; I189; Q191; D134; E188; P38; A132; Q106; and L140. Residues that lie within 6 Å of S105 in SEQ ID No: 2 are: G39; T103; W104; I189; D134; P38; A132; and Q106. Residues that lie within 6 Å of D187 in SEQ ID No: 2 are: D223; I189; Q191; E188; A132; and L140. Residues that lie within 6 Å of H224 in SEQ ID No: 2 are: A225; L278; W104; D223; I189; E188; A132; and Q106.

The present invention relates to isolated lipase variants, comprising an substitution at one or more (e.g., several) positions corresponding to positions S31; G39; A225; T103; L278; W104; T42; D223; I189; Q191; D134; E188; V221; P38H; G41; A132; N79; Q106; and L140 of the mature polypeptide of SEQ ID NO: 2, wherein the variant has improved activity in an amide-bond reaction.

In some embodiments the invention relates to a variant wherein said variant comprises substitution of at least one amino acid residue corresponding to a position in SEQ ID No: 2 that lie within a distance of 10 Å of at least one of the residues of the catalytic triad selected from: G39; A225; T103V/S/A; L278G/K/R/H; W104Y/K/R; T42G/S/Q/H/I/L/M; D223G/N/A/V/H; I189; Q191; D134V/T/I/F/A/S/K/R; E188; V221A/R/K/H; P38H/N/A/G; G41P/S; A132; N79; Q106; L140R/K/H.

In some embodiments the invention relates to a variant wherein the substitution is: G39A; A225I/V/L/M/K/R/S/T/G/H; I189Y/H/Q/N/R/K/A/E/S/T/G; Q191N/A/R/K/H; E188Q/R/D/N/K/H/A; A132S/N/G/H/K/R; N79V/Q/E; or Q106N/Y.

In some embodiments the invention relates to a variant wherein said variant further comprises substitution of at least one amino acid residue corresponding to a position in SEQ ID No: 2 that lie within a distance of 10 Å of at least one of the residues of the catalytic triad selected from: T103G; L278A/V; W104F/Q; T42N/V/A; D134L; G41A.

In some embodiments the invention relates to a variant wherein said variant comprises substitution of at least one amino acid residue corresponding to a position in SEQ ID No: 2 that lie within a distance of 8 Å of at least one of the residues of the catalytic triad selected from: G39; A225; T103V/S/A; L278G/K/R/H; W104Y/K/R; T42G/S/Q/H/l/L/M; D223G/N/A/V/H; I189; Q191; D134V/T/l/F/A/S/K/R; E188; V221A/R/K/H; P38H/N/A/G; G41P/S; A132; Q106; L140R/K/H.

In some embodiments the invention relates to a variant wherein the substitution is: G39A; A225I/V/L/M/K/R/S/T/G/H; I189Y/H/Q/N/R/K/A/E/S/T/G; Q191N/A/R/K/H; E188Q/R/D/N/K/H/A; A132S/N/G/H/K/R; or Q106N/Y.

In some embodiments the invention relates to a variant wherein said variant further comprises substitution of at least one amino acid residue corresponding to a position in SEQ ID No: 2 that lie within a distance of 8 Å of at least one of the residues of the catalytic triad selected from: T103G; L278A/V; W104F/Q; T42N/V/A; D134L; G41A.

In some embodiments the invention relates to a variant wherein said variant comprises substitution of at least one amino acid residue corresponding to a position in SEQ ID No: 2 that lie within a distance of 6 Å of at least one of the residues of the catalytic triad selected from: G39; A225; T103V/S/A; L278G/K/R/H; W104Y/K/R; D223G/N/A/V/H; I189; Q191; D134V/T/I/F/A/S/K/R; E188; P38H/N/A/G; A132; Q106; L140R/K/H.

In some embodiments the invention relates to a variant wherein the substitution is: G39A; A225I/V/L/M/K/R/S/T/G/H; I189Y/H/Q/N/R/K/A/E/S/T/G; Q191N/A/R/K/H; E188Q/R/D/N/K/H/A; A132S/N/G/H/K/R; or Q106N/Y.

In some embodiments the invention relates to a variant wherein said variant further comprises substitution of at least one amino acid residue corresponding to a position in SEQ ID No: 2 that lie within a distance of 6 Å of at least one of the residues of the catalytic triad selected from: T103G; L278A/V; W104F/Q; D134L.

In certain embodiments the invention relates to a variant which compared to the parent lipase comprises a substitution of at least one amino acid residue that leads to a change in geometry i.e. volume or shape of the active site pocket. This may confer an improved binding or better orientation of the substrate, the transition state or residues of the active site pocket.

In some embodiments the invention relates to a variant wherein said variant comprises a substitution of at least one amino acid residue corresponding to a position in SEQ ID No: 2 selected from: G39; A225; T103V/S/A; L278G; W104Y/K/R; T42G/S/Q/H/I/L/M; D223G/N/A/V/H; I189; Q191; D134V/T/I/F/A/S/K/R; E188; V221A/R/K/H; P38H/N/A/G; G41P/S; A132; N79; Q106.

In some embodiments the invention relates to a variant wherein the substitution is: G39A; A225I/V/L/M/K/R/S/T/G/H; I189Y/H/Q/N/R/K/A/E/S/T/G; Q191N/A/R/K/H; E188Q/R/D/N/K/H/A; A132S/N/G/H/K/R; N79V/Q/E; or Q106N/Y.

In some embodiments the invention relates to a variant wherein said variant further comprises substitution of at least one amino acid residue corresponding to a position in SEQ ID No: 2 selected from: T103G; L278A/V; W104F/Q; T42N/V/A; D134L; G41A.

In certain embodiments the invention relates to a variant which compared to the parent lipase comprises a substitution of at least one amino acid residue that leads to a more positive charge of the residue, i.e. a negatively charged residue is substituted with a neutral or a positively charged residue, and a neutral residue is substituted with a positively charged residue. Without being limiting it is believed that the change to a more positive charge affects at least one of the catalytic triad residues through the electrostatic field.

In some embodiments the invention relates to a variant wherein said variant comprises substitution of at least one amino acid residue corresponding to a position in SEQ ID No: 2 selected from: A225; L278K/R/H; W104K/R; T42H; D223G/N/A/V/H; I189; Q191; D134V/T/I/F/A/S/K/R; E188; V221R/K/H; P38; A132; Q106; and L140R/K/H.

In some embodiments the invention relates to a variant wherein the substitution is: A225K/R/H; I189R/K/H; Q191R/K/H; E188Q/R/N/K/H/A; A132H/K/R; and Q106N/Y.

In some embodiments the invention relates to a variant wherein said variant further comprises substitution of at least one amino acid residue corresponding to a position in SEQ ID No: 2 selected from: D134L.

In certain embodiments the invention relates to a variant comprising substitutions of two or more (several) amino acid residues that affect either the geometry i.e. volume or shape of the active site pocket, or the charge of an active site residue or both. In some embodiments the invention relates to a variant wherein at least one substitution that affects the geometry is combined with at least one substitution that affects the charge of the active site residue.

In one aspect, the variant has improved activity in an amide-bond reaction and a sequence identity to the parent lipase of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the amino acid sequence of the variant differs with up to 20 amino acids, e.g., 1, 2, 3, 4, 5, 6, 7, 8, 9, 10, 11, 12, 13, 14, 15, 16, 17, 18, 19, or 20 from the parent lipase.

Parent Lipase

The parent polypeptide i.e. the parent lipase may be obtained from organisms of any genus. For purposes of the present invention, the term “obtained from” as used herein in connection with a given source shall mean that the parent polypeptide encoded by a polynucleotide is produced by the source or by a cell in which the polynucleotide from the source has been inserted. In one aspect, the parent polypeptide is secreted extracellularly.

The parent polypeptide may be a fungal polypeptide. In one aspect, the parent polypeptide is a fungal polypeptide such as a Candida antarctica lipase B, Hyphozyma sp. lipase, Ustilago maydis lipase, Sporisorium reilianum lipase or Cryptococcus tsukubaensis (Pseudozyma tsukubaensis) lipase polypeptide. It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

In one aspect, the parent polypeptide consists or comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to a lipase selected from the group consisting of: Candida antarctica lipase B (SEQ ID No: 2), Hyphozyma sp. lipase (SEQ ID No: 3), Ustilago maydis lipase (SEQ ID No: 4), Sporisorium reilianum lipase, SEQ ID No: 5), and Cryptococcus tsukubaensis (Pseudozyma tsukubaensis) lipase (SEQ ID No: 6).

The parent lipase may be (a) a polypeptide having at least 60% sequence identity to the mature polypeptide of SEQ ID NO: 2; (b) a polypeptide encoded by a polynucleotide that hybridizes under low stringency conditions with (i) the mature polypeptide coding sequence of SEQ ID NO: 1, or (ii) the full-length complement of (i); or (c) a polypeptide encoded by a polynucleotide having at least 60% sequence identity to the mature polypeptide coding sequence of SEQ ID NO: 1.

TABLE 1
Lipase amino acid sequences

| SEQ | | | | Relative |
|---|---|---|---|---|
| ID | | | Amino | sequence |
| No: | Lipase | Source | acids | identity |
| 2 | Candida antarctica | UniProt 1TCA | 317 | 100% |
| | lipase B | | | |
| 3 | Hyphozyma sp. Lipase | WO9324619 | 319 | 73% |
| 4 | Ustilago maydis lipase | UniProt Q4pep1 | 336 | 68% |
| 5 | Sporisorium reilianum | UniProt E6ZUC1 | 341 | 75% |
| | lipase | | | |
| 6 | Cryptococcus | Suen et al., PEDS | 316 | 78% |
| | tsukubaensis | (2004), page | | |
| | (Pseudozyma | 133-140, FIG. 5 | | |
| | tsukubaensis) lipase | | | |

In one aspect, the parent polypeptide has an amino acid sequence that differs with less than 20 amino acids, less than 19 amino acids, less than 18 amino acids, less than 17 amino acids, less than 16 amino acids, less than 15 amino acids, less than 14 amino acids, less than 13 amino acids, less than 12 amino acids, less than 11 amino acids, less than 10 amino acids, less than 9 amino acids, less than 8 amino acids, less than 7 amino acids, less than 6 amino acids, less than 5 amino acids, less than 4 amino acids, less than 3 amino acids, less than 2 amino acids, or with 0 amino acid from the polypeptides of any of SEQ ID No: 2-6.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

In one aspect the parent polypeptide consists or comprises the amino acid sequences of any of SEQ ID No: 2-6, allelic variants thereof or fragments thereof. In one aspect, the parent polypeptide consists or comprises the mature polypeptides of any of SEQ ID No: 2-6. In another aspect, the parent polypeptide consists or comprises amino acids selected from the group consisting of: amino acid 1 to 317 of SEQ ID No: 2; amino acid 1 to 319 of SEQ ID No: 3; amino acid 1 to 336 of SEQ ID No: 4; amino acid 1 to 341 of SEQ ID No: 5; amino acid 1 to 316 of SEQ ID No: 6; allelic variants thereof and fragments thereof.

An allelic variant of the polypeptide or the mature polypeptide of any of SEQ ID No: 2-6 is a polypeptide encoded by an allelic variant, i.e. any of two or more alternative forms of a gene occupying the same chromosomal locus.

A fragment of the polypeptide or the mature polypeptide of any of SEQ ID No: 2-6 is a polypeptide having one or more (several) amino acids deleted from the amino and/or carboxyl terminus of this amino acid sequence. Preferably, a fragment contains at least 100 amino acid residues, at least 125 amino acid residues, at least 150 amino acid residues, at least 175 amino acid residues, at least 180 amino acid residues, at least 190 amino acid residues, at least 200 amino acid residues, at least 210 amino acid residues, at least 220 amino acid residues or at least 230 amino acid residues. The fragment may consist of 100 to 450 amino acids, e.g., 150 to 450, 175 to 400, 200 to 350, 225 to 325, or 250 to 300 amino acids. In one aspect, the parent polypeptide consists or comprises of amino acids residue 34 to 277 or 39 to 254 of SEQ ID No: 2.

A subsequence of the mature polypeptide coding sequence of SEQ ID No: 1, or a homolog thereof, is a nucleotide sequence where one or more (several) nucleotides have been deleted from the 5′- and/or 3′-end. Preferably, a subsequence contains at least 300 nucleotides, at least 375 nucleotides, at least 450 nucleotides, at least 525 nucleotides, at least 540 nucleotides, at least 570 nucleotides, at least 600 nucleotides, at least 630 nucleotides, at least 660 nucleotides, or at least 690 nucleotides.

The polynucleotide of SEQ ID No: 1; or a subsequence thereof; as well as the amino acid sequence of SEQ ID No: 2; or a fragment thereof; may be used to design nucleic acid probes to identify and clone DNA encoding parent polypeptides from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, at least 25, at least 35, or at least 70 nucleotides in length. It is however, preferred that the nucleic acid probe is at least 100 nucleotides in length. For example, the nucleic acid probe may be at least 200 nucleotides, at least 300 nucleotides, at least 400 nucleotides, at least 500 nucleotides, or at least 600 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other organisms may be screened for DNA that hybridizes with the probes described above and encodes a parent polypeptide. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID No: 1, or a subsequence thereof, the carrier material is used in a Southern blot.

For purposes of the present invention, hybridization indicates that the polynucleotide hybridizes to a labeled nucleic acid probe corresponding to (i) SEQ ID NO: 1; (ii) the mature polypeptide coding sequence of SEQ ID NO: 1; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of SEQ ID No: 1. In another aspect, the nucleic acid probe is nucleotides 76 to 1026 of SEQ ID No: 1. In another aspect, the nucleic acid probe is a polynucleotide sequence that encodes the polypeptide of SEQ ID No: 2, or a subsequence thereof.

For long probes of at least 100 nucleotides in length, very low to very high stringency conditions are defined as prehybridization and hybridization at 42° C. in 5× SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2× SSC, 0.2% SDS at 45° C. (very low stringency), at 50° C. (low stringency), at 55° C. (medium stringency), at 60° C. (medium-high stringency), at 65° C. (high stringency), or at 70° C. (very high stringency).

For short probes that are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, PNAS USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally.

For short probes that are about 15 nucleotides to about 70 nucleotides in length, the carrier material is washed once in 6× SCC plus 0.1% SDS for 15 minutes and twice each for 15 minutes using 6× SSC at 5° C. to 10° C. below the calculated Tₘ.

In a third aspect, the parent polypeptide is encoded by a polynucleotide comprising or consisting of a nucleotide sequence with a degree of identity to the mature polypeptide coding sequence of SEQ ID No: 1 of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the mature polypeptide coding sequence is nucleotides 76 to 1026 of SEQ ID No: 1.

In another aspect, the parent is encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, or (ii) the full-length complement of (i) (Sambrook et al., 1989, Molecular Cloning, A Laboratory Manual, 2d edition, Cold Spring Harbor, New York).

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

Variants

The present invention provides lipase variants, comprising a substitution at one or more (e.g., several) positions corresponding to positions S31; G39; A225; T103; L278; W104; T42; D223; I189; Q191; D134 E188; V221; P38H; G41; A132; N79; Q106; and L140 of SEQ ID No: 2, wherein the variant has improved activity in an amide-bond reaction.

In an embodiment, the variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent lipase.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the mature polypeptide of SEQ ID NO: 2. In one embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the amino acid sequence 34-277 of SEQ ID NO: 2. In one embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the amino acid sequence 38-254 of SEQ ID NO: 2.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the mature polypeptide of SEQ ID NO: 3.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the mature polypeptide of SEQ ID NO: 4.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the mature polypeptide of SEQ ID NO: 5.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the mature polypeptide of SEQ ID NO: 6.

In one aspect, the number of substitutions in the variants of the present invention is 1-20, 1-10, 1-5, 1, 2, 3, 4, 5, 6, 7, 8, 9, 10, 11, 12, 13, 14, 15, 16, 17, 18, 19, or 20 alterations.

In another aspect, a variant comprises a substitution at one or more (e.g., several) positions corresponding to positions S31; G39; A225; T103; L278; W104; T42; D223; I189; Q191; D134; E188; V221; P38H; G41; A132; N79; Q106; and L140. In another aspect, a variant comprises a substitution at two positions corresponding to any of positions S31; G39; A225; T103; L278; W104; T42; D223; I189; Q191; D134; E188; V221; P38H; G41; A132; N79; Q106; and L140. In another aspect, a variant comprises a substitution at three positions corresponding to any of positions S31; G39; A225; T103; L278; W104; T42; D223; I189; Q191; D134; E188; V221; P38H; G41; A132; N79; Q106; and L140. In another aspect, a variant comprises a substitution at four positions corresponding to any of positions S31; G39; A225; T103; L278; W104; T42; D223; I189; Q191; D134; E188; V221; P38H; G41; A132; N79; Q106; and L140. In another aspect, a variant comprises a substitution at five positions corresponding to any of positions S31; G39; A225; T103; L278; W104; T42; D223; I189; Q191; D134; E188; V221; P38H; G41; A132; N79; Q106; and L140. In another aspect, a variant comprises a substitution at six positions corresponding to any of positions S31; G39; A225; T103; L278; W104; T42; D223; I189; Q191; D134; E188; V221; P38H; G41; A132; N79; Q106; and L140. In another aspect, a variant comprises a substitution at seven positions corresponding to any of positions S31; G39; A225; T103; L278; W104; T42; D223; I189; Q191; D134; E188; V221; P38H; G41; A132; N79; Q106; and L140. In another aspect, a variant comprises a substitution at eight positions corresponding to any of positions S31; G39; A225; T103; L278; W104; T42; D223; I189; Q191; D134; E188; V221; P38H; G41; A132; N79; Q106; and L140. In another aspect, a variant comprises a substitution at nine positions corresponding to any of positions S31; G39; A225; T103; L278; W104; T42; D223; I189; Q191; D134; E188; V221; P38H; G41; A132; N79; Q106; and L140. In another aspect, a variant comprises a substitution at ten positions corresponding to any of positions S31; G39; A225; T103; L278; W104; T42; D223; I189; Q191; D134; E188; V221; P38H; G41; A132; N79; Q106; and L140. In another aspect, a variant comprises a substitution at eleven positions corresponding to any of positions S31; G39; A225; T103; L278; W104; T42; D223; I189; Q191; D134; E188; V221; P38H; G41; A132; N79; Q106; and L140. In another aspect, a variant comprises a substitution at twelve positions corresponding to any of positions S31; G39; A225; T103; L278; W104; T42; D223; I189; Q191; D134; E188; V221; P38H; G41; A132; N79; Q106; and L140. In another aspect, a variant comprises a substitution at thirteen positions corresponding to any of positions S31; G39; A225; T103; L278; W104; T42; D223; I189; Q191; D134; E188; V221; P38H; G41; A132; N79; Q106; and L140. In another aspect, a variant comprises a substitution at fourteen positions corresponding to any of positions S31; G39; A225; T103; L278; W104; T42; D223; I189; Q191; D134; E188; V221; P38H; G41; A132; N79; Q106; and L140. In another aspect, a variant comprises a substitution at fifteen positions corresponding to any of positions S31; G39; A225; T103; L278; W104; T42; D223; I189; Q191; D134; E188; V221; P38H; G41; A132; N79; Q106; and L140. In another aspect, a variant comprises a substitution at sixteen positions corresponding to any of positions S31; G39; A225; T103; L278; W104; T42; D223; I189; Q191; D134; E188; V221; P38H; G41; A132; N79; Q106; and L140. In another aspect, a variant comprises a substitution at seventeen positions corresponding to any of positions S31; G39; A225; T103; L278; W104; T42; D223; I189; Q191; D134; E188; V221; P38H; G41; A132; N79; Q106; and L140. In another aspect, a variant comprises a substitution at eighteen positions corresponding to any of positions S31; G39; A225; T103; L278; W104; T42; D223; I189; Q191; D134; E188; V221; P38H; G41; A132; N79; Q106; and L140. In another aspect, a variant comprises a substitution at each position corresponding to positions S31; G39; A225; T103; L278; W104; T42; D223; I189; Q191; D134; E188; V221; P38H; G41; A132; N79; Q106; and L140.

In another aspect, the variant comprises or consists of a substitution at a position corresponding to position S31. In another aspect, the amino acid at a position corresponding to position S31 is substituted with Arg. In another aspect, the variant comprises or consists of the substitution S31R or S31K of the mature polypeptide of SEQ ID NO: 2.

In another aspect, the variant comprises or consists of a substitution at a position corresponding to position G39. In another aspect, the amino acid at a position corresponding to position G39 is substituted with Ala. In another aspect, the variant comprises or consists of the substitution G39A of the mature polypeptide of SEQ ID NO: 2.

In another aspect, the variant comprises or consists of a substitution at a position corresponding to position A225. In another aspect, the amino acid at a position corresponding to position A225 is substituted with Ile, Val, Leu, Met, Lys, Arg, Ser, Thr, Gly, or His. In another aspect, the variant comprises or consists of the substitution A225I, A225V, A225L, A225M, A225K, A225R, A225S, A225T, A225G, or A225H of the mature polypeptide of SEQ ID NO: 2.

In another aspect, the variant comprises or consists of a substitution at a position corresponding to position T103. In another aspect, the amino acid at a position corresponding to position T103 is substituted with Ala, Ser, or Val. In another aspect, the variant comprises or consists of the substitution T103V, T103S, or T103A of the mature polypeptide of SEQ ID NO: 2.

In another aspect, the variant comprises or consists of a substitution at a position corresponding to position L278. In another aspect, the amino acid at a position corresponding to position L278 is substituted with Arg, Gly, His, or Lys. In another aspect, the variant comprises or consists of the substitution L278G, L278K, L278R, or L278H of the mature polypeptide of SEQ ID NO: 2.

In another aspect, the variant comprises or consists of a substitution at a position corresponding to position W104. In another aspect, the amino acid at a position corresponding to position W104 is substituted with Arg, Lys, or Tyr. In another aspect, the variant comprises or consists of the substitution W104Y, W104K, or W104R of the mature polypeptide of SEQ ID NO: 2.

In another aspect, the variant comprises or consists of a substitution at a position corresponding to position T42. In another aspect, the amino acid at a position corresponding to position T42 is substituted with Gln, Gly, His, Ile, Leu, Met, or Ser. In another aspect, the variant comprises or consists of the substitution T42G, T42S, T42Q, T42H, T42I, T42L, or T42M of the mature polypeptide of SEQ ID NO: 2.

In another aspect, the variant comprises or consists of a substitution at a position corresponding to position D223. In another aspect, the amino acid at a position corresponding to position D223 is substituted with Ala, Asn, Gly, His, or Val. In another aspect, the variant comprises or consists of the substitution D223G, D223N, D223A, D223V, or D223H of the mature polypeptide of SEQ ID NO: 2.

In another aspect, the variant comprises or consists of a substitution at a position corresponding to position I189. In another aspect, the amino acid at a position corresponding to position I189 is substituted with Ala, Arg, Asn, Gln, Glu, His, Lys, Ser, Thr, or Val. In another aspect, the variant comprises or consists of the substitution I189Y, I189H, I189Q, I189N, I189R, I189K, I189A, I189E, I189S, I189T, or I189G of the mature polypeptide of SEQ ID NO: 2.

In another aspect, the variant comprises or consists of a substitution at a position corresponding to position Q191. In another aspect, the amino acid at a position corresponding to position Q191 is substituted with Asn, Ala, Arg, Lys, or His. In another aspect, the variant comprises or consists of the substitution Q191N, Q191A, Q191R, Q191K, or Q191H of the mature polypeptide of SEQ ID NO: 2.

In another aspect, the variant comprises or consists of a substitution at a position corresponding to position D134. In another aspect, the amino acid at a position corresponding to position D134 is substituted with Ala, Arg, Ile, Lys, Phe, Ser, Thr, or Val. In another aspect, the variant comprises or consists of the substitution D134V, D134T, D134I, D134F, D134A, D134S, D134K, or D134R of the mature polypeptide of SEQ ID NO: 2.

In another aspect, the variant comprises or consists of a substitution at a position corresponding to position E188. In another aspect, the amino acid at a position corresponding to position E188 is substituted with Gln, Arg, Asp, Asn, Lys, His, or Ala. In another aspect, the variant comprises or consists of the substitution E188Q, E188R, E188D, E188N, E188K, E188H, or E188A of the mature polypeptide of SEQ ID NO: 2.

In another aspect, the variant comprises or consists of a substitution at a position corresponding to position V221. In another aspect, the amino acid at a position corresponding to position V221 is substituted with Ala, Arg, His, or Lys. In another aspect, the variant comprises or consists of the substitution V221A, V221R, V221K, or V221H of the mature polypeptide of SEQ ID NO: 2.

In another aspect, the variant comprises or consists of a substitution at a position corresponding to position P38. In another aspect, the amino acid at a position corresponding to position P38 is substituted with Ala, Asn, Gly, or His. In another aspect, the variant comprises or consists of the substitution P38H, P38N, P38A, or P38G of the mature polypeptide of SEQ ID NO: 2.

In another aspect, the variant comprises or consists of a substitution at a position corresponding to position G41. In another aspect, the amino acid at a position corresponding to position G41 is substituted with Pro, or Ser. In another aspect, the variant comprises or consists of the substitution G41P, or G41S of the mature polypeptide of SEQ ID NO: 2.

In another aspect, the variant comprises or consists of a substitution at a position corresponding to position A132. In another aspect, the amino acid at a position corresponding to position A132 is substituted with Ser, Asn, Gly, His, Lys, or Arg. In another aspect, the variant comprises or consists of the substitution A132S, A132N, A132G, A132H, A132K, or A132R of the mature polypeptide of SEQ ID NO: 2.

In another aspect, the variant comprises or consists of a substitution at a position corresponding to position N79. In another aspect, the amino acid at a position corresponding to position N79 is substituted with Val, Gin, or Glu. In another aspect, the variant comprises or consists of the substitution N79V, N79Q, or N79E of the mature polypeptide of SEQ ID NO: 2.

In another aspect, the variant comprises or consists of a substitution at a position corresponding to position Q106. In another aspect, the amino acid at a position corresponding to position Q106 is substituted with Asn, or Tyr. In another aspect, the variant comprises or consists of the substitution Q106N, or Q106Y of the mature polypeptide of SEQ ID NO: 2.

In another aspect, the variant comprises or consists of a substitution at a position corresponding to position L140. In another aspect, the amino acid at a position corresponding to position L140 is substituted with Arg, His, or Lys. In another aspect, the variant comprises or consists of the substitution L140R, L140K, or L140H of the mature polypeptide of SEQ ID NO: 2.

In another aspect, the variant comprises or consists of any of the following combination of substitutions of amino acid residues corresponding to a position in SEQ ID No: 2 as shown in table 2 below:

TABLE 2
Substitutions of amino acid residues corresponding to a position in SEQ ID No: 2

| No | Variant |
|---|---|
| 1 | A225K |
| 2 | A225I |
| 3 | A225V |
| 4 | A225L |
| 5 | A225M |
| 6 | A225S |
| 7 | G39A |
| 8 | D223G |
| 9 | D223N |
| 10 | T42G |
| 11 | T42S |
| 12 | Q191R |
| 13 | I189Y |
| 14 | I189H |
| 15 | T103V |
| 16 | T103S |
| 17 | T103A |
| 18 | G39A + L278A |
| 19 | G39A + W104F |
| 20 | G39A + Q191R |
| 21 | G39A + L278A + A281G |
| 22 | G39A + T103G + L278A |
| 23 | G39A + W104F + L278A |
| 24 | G39A + W104Q + L278A |
| 25 | G39A + T103G + W104F + L278A |
| 26 | G39A + T103G + W104Q + L278A |
| 27 | G39A + T103G + W104Y + L278A |
| 28 | G39A + T103G + W104F + D223G + A225K + L278A |
| 29 | G39A + T103G + W104F + D223N + A225K + L278A |
| 30 | G39A + W104F + A225K + L278A |
| 31 | G39A + W104F + D223N + A225K + L278A |
| 32 | G39A + W104F + D223N + A225I + L278A |
| 33 | G39A + T103G + D223N + A225K + L278A |
| 34 | G39A + T103G + W104Y + D223N + A225K + L278A |
| 35 | G39A + T103G + W104F + M129L + L278A |
| 36 | G39A + T103G + W104F + M129L + D223N + L278A |
| 37 | G39A + A225K + L278A |
| 38 | G39A + D223G + A225K + L278A |
| 39 | G39A + D223G + A225I + L278A |
| 40 | G39A + T103G + W104F + A225K + L278A |
| 41 | G39A + T103G + W104F + A225I + L278A |
| 42 | G39A + T103G + W104F + D223G + A225I + L278A |
| 43 | G39A + T103G + W104F + D223N + A225I + L278A |
| 44 | G39A + W104F + A225I + L278A |
| 45 | G39A + W104F + V221R + D223N + A225K + L278A |
| 46 | G39A + T103G + A225K + L278A |
| 47 | G39A + T103G + A225I + L278A |
| 48 | G39A + T103G + D223G + A225K + L278A |
| 49 | G39A + T103G + D223G + A225I + L278A |
| 50 | G39A + T103G + D223N + A225I + L278A |
| 51 | G39A + T103G + I189Y + L278A |
| 52 | G39A + T103G + W104Y + I189Y + D223N + A225K + L278A |
| 53 | G39A + W104F + I189Y + D223N + A225K + L278A |
| 54 | G39A + T103G + W104F + V221R + D223N + A225K + L278A |
| 55 | G39A + T103G + I189Y + D223N + A225K + L278A |
| 56 | G39A + T103G + W104Y + I189Y + L278A |
| 57 | T103G + W104F + D223G + A225K |
| 58 | T103G + W104F + A225K |
| 59 | T103G + W104F + I189Y + D223G |
| 60 | T103G + W104F + I189Y |
| 61 | T103G + W104F + D223G + L278A |
| 62 | T103G + W104F + L278A |
| 63 | T103G + W104F + Q191R + D223G |
| 64 | T103G + W104F + Q191R |
| 65 | G39A + T42G + T103G + W104F + I189Y + D223G + A225K |
| 66 | G39A + T42G + T103G + W104F + D223G + A225K |
| 67 | G39A + T42G + T103G + W104F + I189Y + Q191R + D223G + A225K |
| 68 | G39A + T42G + T103G + W104F + Q191R + D223G + A225K |
| 69 | G39A + W104F + D223G + A225K |
| 70 | G39A + W104F + I189Y + D223G + A225K |
| 71 | G39A + W104F + I189Y + A225K |
| 72 | G39A + W104F + A225K |
| 73 | G39A + W104F + I189H |
| 74 | G39A + W104F + I189Y + D223G |
| 75 | G39A + W104F + I189Y |
| 76 | G39A + W104F + D223G + L278A |
| 77 | G39A + W104F + I189Y + D223G + L278A |
| 78 | G39A + W104F + I189Y + L278A |
| 79 | G39A + W104F + L278A |
| 80 | G39A + T42G + T103G + W104F + M129L + I189Y + D223G + A225K + L278A |
| 81 | G39A + T103G + W104F + D223G |
| 82 | G39A + T103G + W104F + I189Y + D223G |
| 83 | G39A + T103G + W104F + I189Y |
| 84 | G39A + T103G + W104F |
| 85 | G39A + W104F + I189Y + A281G + A282G + I285A + V286A + L278A |
| 86 | G39A + W104F + A281G + A282G + I285A + V286A + L278A |
| 87 | W104F + I189Y + A281G + A282G + I285A + V286A + L278A |
| 88 | W104F + L278A + A281G + A282G + I285A + V286A |
| 89 | G39A + W104F + Q191R |
| 90 | G39A + W104F + Q191R + D223G |
| 91 | G39A + T42G + T103G + W104F + D223G + A225K + L278A |
| 92 | G39A + T42G + T103G + W104F + D223N + A225K + L278A |
| 93 | G39A + T42G + W104F + A225K + L278A |
| 94 | G39A + T42G + W104F + D223N + A225K + L278A |
| 95 | G39A + T42G + W104F + D223N + A225I + L278A |
| 96 | G39A + T42G + T103G + D223N + A225K + L278A |
| 97 | G39A + T42G + T103G + W104Y + D223N + A225K + L278A |
| 98 | T42G + T103G + W104F + D223G + A225K + L278A |
| 99 | T42G + T103G + W104F + D223N + A225K + L278A |
| 100 | T42G + W104F + A225K + L278A |
| 101 | T42G + W104F + D223N + A225K + L278A |
| 102 | T42G + W104F + D223N + A225I + L278A |
| 103 | T42G + T103G + D223N + A225K + L278A |
| 104 | T42G + T103G + W104Y + D223N + A225K + L278A |
| 105 | G39A + T103G + W104F + I189Y + L278A |
| 106 | G39A + T103G + W104F + D223G + L278A |
| 107 | S31R + G39A + T103G + W104F + L278A |
| 108 | I189A |
| 109 | S31K |
| 110 | S31R |
| 111 | I189G |
| 112 | S31R + G39A + L278A |

The variants may further comprise one or more additional alterations at one or more (e.g., several) other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Asn/Gln, Thr/Ser, Ala/Gly, Ala/Thr, Ser/Asn, Ala/Val, Ser/Gly, Tyr/Phe, Ala/Pro, Lys/Arg, Asp/Asn, Glu/Gln, Leu/Ile, Leu/Val, Ala/Glu, and Asp/Gly.

In an embodiment, the variant has improved catalytic efficiency compared to the parent lipase. In an embodiment, the variant has improved catalytic rate compared to the parent lipase. In an embodiment, the variant has improved specific activity compared to the parent lipase. In an embodiment, the variant has improved substrate binding compared to the parent lipase. In an embodiment, the variant has improved substrate cleavage compared to the parent lipase. In an embodiment, the variant has improved substrate specificity compared to the parent lipase. In an embodiment, the variant has improved product release compared to the parent lipase. In an embodiment, the variant has improved turnover number compared to the parent lipase.

Preparation of Variants

The present invention also relates to methods for obtaining a variant with improved activity in amide-bond reaction, comprising: (a) introducing into a parent lipase a substitution at one or more (e.g., several) positions corresponding to positions S31; G39; A225; T103; L278; W104; T42; D223; I189; Q191; D134; E188; V221; P38; G41; A132; N79; Q106; and L140 of the mature polypeptide of SEQ ID NO: 2, wherein the variant has improved activity in amide-bond reaction; and (b) recovering the variant.

The variants can be prepared using any mutagenesis procedure known in the art, such as site-directed mutagenesis, synthetic gene construction, semi-synthetic gene construction, random mutagenesis, shuffling, mutator strains, physical and chemical mutagenes (e.g. UV-light, ionizing radiation, nitrous acid, hydroxylamine), etc.

Site-directed mutagenesis is a technique in which one or more (e.g., several) mutations are introduced at one or more defined sites in a polynucleotide encoding the parent. Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966. Site-directed mutagenesis can also be accomplished in vivo by methods known in the art. See, e.g., US2004/0171154; Storici et al., 2001, Nature Biotechnol. 19: 773-776; Kren et al., 1998, Nat. Med. 4: 285-290; and Calissano & Macino, 1996, Fungal Genet. Newslett. 43: 15-16. Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO95/17413; or WO95/22625. Other methods that can be used include error-prone PCR, phage display (e.g., Lowman et al., 1991, Biochemistry 30: 10832-10837; U.S. Pat. No. 5,223,409; WO92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

Polynucleotides

The present invention also relates to isolated polynucleotides encoding a variant of the present invention.

Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO96/00787), Fusarium venenatum amyloglucosidase (WO00/56900), Fusarium venenatum Dania (WO00/56900), Fusarium venenatum Quinn (WO00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator sequence is operably linked to the 3′-terminus of the polynucleotide encoding the variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB). Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease. Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader sequence is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked with the regulatory sequence.

Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO00/24883). Isolation of the AMA1 gene and construction of plasmids or vectors comprising the gene can be accomplished according to the methods disclosed in WO00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804) or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980). The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known in the art. Suitable procedures for transformation of Aspergillus and Trichoderma host cells are described in EP238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

Methods of Production

The present invention also relates to methods of producing a variant, comprising: (a) cultivating a host cell of the present invention under conditions suitable for expression of the variant; and (b) recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

Uses

The lipase variant of the invention may be used in an amide-bond reaction, such as in amide synthesis, hydrolysis, alcoholysis, acidolysis, aminolysis, transamidification, transacylation or acyl transfer. An application of the lipase variant may be in the field of biocatalysis for the purpose of organic synthesis, analysis or degradation, e.g. removal of protection groups or racemic resolution of amides, amines, carboxylic acids, esters and alcohols. The lipase variant may particularly be applied for its stability, ability to accept a relative broad range of substrates, chemo-, regio-, and/or stereo-selectivity. The lipase variant may be used immobilized, in aqueous solutions, in buffered solutions, in organic solvents, in solvent-free liquid substrates, in gas-phase, in ionic liquids or in supercritical fluids. The reaction products may be used in the bulk chemical, fine chemical, agrochemical, pharmaceutical, food or feed industry.

Further examples for the use of the lipase variant are degradation of unwanted amides, e.g. decontamination of soil, surfaces, food or feed from e.g. pesticides like the herbicides beflubutamid, benzipram, bromobutide or tebutam. The lipase variant may also be used for the degradation of naturally occurring amides, e.g. capsaicinoids contained in chillies and other Capsicum species for reduction of pungency. The lipase variant may be applied in the modifications of products containing amides, e.g. nylon.

Plants

The present invention also relates to plants, e.g., a transgenic plant, plant part, or plant cell, comprising a polynucleotide of the present invention so as to express and produce the variant in recoverable quantities. The variant may be recovered from the plant or plant part. Alternatively, the plant or plant part containing the variant may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn). Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats.

Also included within the scope of the present invention are the progeny of such plants, plant parts, and plant cells.

The transgenic plant or plant cell expressing a variant may be constructed in accordance with methods known in the art. In short, the plant or plant cell is constructed by incorporating one or more expression constructs encoding a variant into the plant host genome or chloroplast genome and propagating the resulting modified plant or plant cell into a transgenic plant or plant cell.

The expression construct is conveniently a nucleic acid construct that comprises a polynucleotide encoding a variant operably linked with appropriate regulatory sequences required for expression of the polynucleotide in the plant or plant part of choice. Furthermore, the expression construct may comprise a selectable marker useful for identifying plant cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the variant is desired to be expressed. For instance, the expression of the gene encoding a variant may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pin2 promoter (Xu et al., 1993, Plant Mol. Biol. 22: 573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a variant in the plant. For instance, the promoter enhancer element may be an intron that is placed between the promoter and the polynucleotide encoding a variant. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mol. Biol. 21: 415-428. Additional transformation methods include those described in U.S. Pat. Nos. 6,395,966 and 7,151,204 (both of which are herein incorporated by reference in their entirety).

Following transformation, the transformants having incorporated the expression construct are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase.

In addition to direct transformation of a particular plant genotype with a construct of the present invention, transgenic plants may be made by crossing a plant having the construct to a second plant lacking the construct. For example, a construct encoding a variant can be introduced into a particular plant variety by crossing, without the need for ever directly transforming a plant of that given variety. Therefore, the present invention encompasses not only a plant directly regenerated from cells which have been transformed in accordance with the present invention, but also the progeny of such plants. As used herein, progeny may refer to the offspring of any generation of a parent plant prepared in accordance with the present invention. Such progeny may include a DNA construct prepared in accordance with the present invention. Crossing results in the introduction of a transgene into a plant line by cross pollinating a starting line with a donor plant line. Non-limiting examples of such steps are described in U.S. Pat. No. 7,151,204.

Plants may be generated through a process of backcross conversion. For example, plants include plants referred to as a backcross converted genotype, line, inbred, or hybrid.

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The present invention also relates to methods of producing a variant of the present invention comprising: (a) cultivating a transgenic plant or a plant cell comprising a polynucleotide encoding the variant under conditions conducive for production of the variant; and (b) recovering the variant.

### EXAMPLES

The present invention is further described by the following examples that should not be construed as limiting the scope of the invention.

### Example 1

### Generation of Variants

(A) To generate variants of SEQ ID No: 1 for expression in an Aspergillus oryzae strain, PCR-based site-directed mutagenesis was done with mutagenic primers that introduce the desired sequence change (substitutions). Primers were designed so that the mutation lies in the middle of the oligonucleotide with sufficient flanking nucleotides (15-25). The PCR template was a 7467 base pair plasmid that contained the SEQ ID No: 1 and that can be propagated in E. coli and can transform Aspergillus strains. The PCR was setup with a proofreading DNA polymerase (Phusion DNA polymerase (from Finnzymes, Themo Scientific), alternatively KOD DNA polymerase (from Novagen, Toyobo)).

The PCR products were used to transform competent E. coli DH5 α cells (from TaKaRa) according to the instructions from the manufacturer. Plasmid DNA was isolated from monoclonal transformed E. coli strains, and sequenced to verify the presence of the desired substitution. Confirmed plasmid variants were used to transform an Aspergillus oryzae strain that is negative in pyrG (orotidine-5′-phosphate decarboxylase) and that is also negative in the proteases pepC (a serine protease homologous to yscB), alp (an alkaline protease) Npl (a neutral metalloprotease I) to avoid degradation of the lipase variants during and after fermentation.

The transformed Aspergillus strains were fermented as submerged culture in shake flasks and the lipase variants secreted into the fermentation medium. After the fermentation, the lipase variants were purified from the sterile filtered fermentation medium in a 3 step procedure with (1) hydrophobic interaction chromatography on decylamine-agarose, alternatively on butyl-toyopearl, (2) buffer exchange by gel filtration, alternatively by ultra-filtration and (3) ion exchange chromatography with cation exchange on SP-sepharose at pH 4.5, alternatively with anion exchange on Q-sepharose at pH 7. The lipase variant solutions were stored frozen.

(B) To generate variants of SEQ ID No: 1 for expression in a Pichia pastoris strain, PCR-based site-directed mutagenesis was done with mutagenic primers that introduced the desired sequence change (substitutions). Primers were designed so that the mutation lies in the middle of the oligonucleotide with sufficient flanking nucleotides (15-25). The PCR template was a 8604 basepair plasmid that contained the SEQ ID No: 1 with a His-tag and that can be propagated in E. coli and can transform in Pichia strains. The PCR was setup with a proofreading DNA polymerase (Phusion DNA polymerase (from Finnzymes, Themo Scientific).

The PCR products were used to transform competent E. coli DH5 α cells (from TaKaRa) according to the instructions from the manufacturer. Plasmid DNA was isolated from monoclonal transformed E. coli strains, and sequenced to verify the presence of the desired substitution. Confirmed plasmid variants were used to transform an Pichia pastoris strain that is Mut(s), Suc(+), His(−).

The transformed Pichia strains were fermented as submerged culture in deep well plates and secretion of the lipase variants into the fermentation medium were induced by addition of methanol. After the fermentation, the lipase variants were purified from the cleared supernants using a standard His-tag purification protocol (from Qiagen) and buffer-exchanged into 50 mM phosphate buffer, pH 7.0 using Amicon Ultra centrifugal filter devices with a 10 kDa cut-off (from Merck Millipore).

with a C-terminal His-tag (underlined):
SEQ ID NO: 1
ATGAAGCTAC TCTCTCTGAC CGGTGTGGCT GGTGTGCTTG CGACTTGCGT TGCAGCCACT
CCTTTGGTGA AGCGTCTACC TTCCGGTTCG GACCCTGCCT TTTCGCAGCC CAAGTCGGTG
CTCGATGCGG GTCTGACCTG CCAGGGTGCT TCGCCATCCT CGGTCTCCAA ACCCATCCTT
CTCGTCCCCG GAACCGGCAC CACAGGTCCA CAGTCGTTCG ACTCGAACTG GATCCCCCTC
TCAACGCAGT TGGGTTACAC ACCCTGCTGG ATCTCACCCC CGCCGTTCAT GCTCAACGAC
ACCCAGGTCA ACACGGAGTA CATGGTCAAC GCCATCACCG CGCTCTACGC TGGTTCGGGC
AACAACAAGC TTCCCGTGCT TACCTGGTCC CAGGGTGGTC TGGTTGCACA GTGGGGTCTG
ACCTTCTTCC CCAGTATCAG GTCCAAGGTC GATCGACTTA TGGCCTTTGC GCCCGACTAC
AAGGGCACCG TCCTCGCCGG CCCTCTCGAT GCACTCGCGG TTAGTGCACC CTCCGTATGG
CAGCAAACCA CCGGTTCGGC ACTCACCACC GCACTCCGAA ACGCAGGTGG TCTGACCCAG
ATCGTGCCCA CCACCAACCT CTACTCGGCG ACCGACGAGA TCGTTCAGCC TCAGGTGTCC
AACTCGCCAC TCGACTCATC CTACCTCTTC AACGGAAAGA ACGTCCAGGC ACAGGCCGTG
TGTGGGCCGC TGTTCGTCAT CGACCATGCA GGCTCGCTCA CCTCGCAGTT CTCCTACGTC
GTCGGTCGAT CCGCCCTGCG CTCCACCACG GGCCAGGCTC GTAGTGCAGA CTATGGCATT
ACGGACTGCA ACCCTCTTCC CGCCAATGAT CTGACTCCCG AGCAAAAGGT CGCCGCGGCT
GCGCTCCTGG CGCCGGCAGC TGCAGCCATC GTGGCGGGTC CAAAGCAGAA CTGCGAGCCC
GACCTCATGC CCTACGCCCG CCCCTTTGCA GTAGGCAAAA GGACCTGCTC CGGCATCGTC
ACCCCCCACC ATCACCATCA CCATTGA

### Example 2

### Determination of Activity in Amide-Bond Reaction

The activity in amide-bond reaction was determined with a fluorimetric assay similar to the one described by Henke E. & Bornscheuer U. T. (2003) Anal Chem., 75, 255-260.

The concentrations in the aqueous reaction mixture were 0.03 mg/ml enzyme, 5 mM substrate, 25 mM phosphate pH 7.0, 10% (w/v) tetrahydrofuran. This reaction mixture was prepared by pipetting 75 uL enzyme solution (that contained 0.08 mg/ml enzyme (lipase variant)) with 125 uL assay solution (that contained 8 mM substrate (benzyl chloroacetamide=2-chloro-N-benzylacetamide), 40 mM phosphate (potassium salt) pH 7.0, 16% (w/v) tetrahydrofuran) in a micotiter well of a 96 wells microtiter plate.

The enzyme solution was prepared by diluting a concentrated enzyme stock solution, whose concentration was determined by measuring the absorbance at 280 nm and calculating the concentration. An extinction coefficient of 1.21 (an absorbance of 1.21 equals 1 mg/ml) was used for Candida antarctica lipase B.

The microtiter plate was covered with Parafilm (from by Pechiney Plastic Packaging Company in Chicago, Ill., USA) and incubated for 18 hours at 37° C. and 300 rpm in the MTP Thermomixer Comfort (form Eppendorf AG in Hamburg, Germany). Afterwards 50 uL 20 mM 4-chloro-7-nitrobenzofurazan (NBD-Cl, 4-chloro-7-nitro-1,2,3-benzoxadiazole) in 1-hexanol was pipetted into the well and incubated for 1 hour at 37° C. and 500 rpm.

Afterwards the fluorescence intensity was measured with the fluorimeter Fluostar Optima (from BMG Labtech GmbH in Ortenberg, Germany) with excitation at 485 nm (filter BMG 0038 485-P) and emission at 540 nm (filter BMG 0414A 540-20) and 100 flashes per well. Each enzyme variant was measured in 3 replications, in 3 wells. In parallel, each enzyme variant was also setup in 3 different wells without substrate, to measure the small background fluorescents from each variant. To measure the small autohydrolysis of the substrate, further 3 wells were setup with substrate and without enzyme. Further 3 wells were setup without substrate and without enzyme, thus only aqueous medium with buffer and solvent, to measure the small background fluorescents from the medium.

The average value from the measurements with enzyme and with substrate is named “es”; the value with enzyme and without substrate is “e”; the value without enzyme and with substrate is “s”; the value without enzyme and without substrate is “o”. To correct for fluorescents from enzyme, medium and autohydrolysis the following subtractions were calculated: (es−e)−(s−o)=(es−s)−(e−o)=es−e−s+o=corrected enzyme variant activity value. s−o=autohydrolysis of substrate. Because these measurements are in arbitrary fluorescents units, these values were normalized to percentage of substrate that reacted. 0% means no reaction of substrate; 100% means complete reaction of substrate to products. For this normalization 2 wells for each enzyme variant were setup in parallel. These wells contained the same composition as the other wells, except that they contained instead of substrate the products at a concentration that corresponds to 10% reaction. In this example, the concentrations in these 2 wells were 0.03 mg/ml enzyme, 0.5 mM hydrolysis products (that is 0.5 mM benzylamine and 0.5 mM chloroacetic acid), 25 mM phosphate pH 7.0, 10% (w/v) tetrahydrofuran. The average value from the measurements with enzyme and with product is named “ep”. To correct for background fluorescents from the enzyme variants, the average value from the measurements with enzyme and without product (and without substrate), which was named “e”, was subtracted. In short, the normalized and corrected enzyme variant activity was calculated as 10·((es−e)−(s−o))/(ep−e).

### 96 Wells Microtiter Plate Setup Schema:

| | | without | | wild- | vari- | vari- | vari- | vari- | vari- | vari- | vari- | vari- | wild- |
|---|---|---|---|---|---|---|---|---|---|---|---|---|---|
| | | enzyme | | type | ant 1 | ant 2 | ant 3 | ant 4 | ant 5 | ant 6 | ant 7 | ant 8 | type |
| | | 1 | 2 | 3 | 4 | 5 | 6 | 7 | 8 | 9 | 10 | 11 | 12 |
| without | A | O | o | e_{wt} | e₁ | e₂ | e₃ | e₄ | e₅ | e₆ | e₇ | e₈ | e_{wt} |
| sub- | B | | | | | | | | | | | | |
| strate | C | | | | | | | | | | | | |
| with | D | s | s | es_{wt} | es₁ | es₂ | es₃ | es₄ | es₅ | es₆ | es₇ | es₈ | es_{wt} |
| sub- | E | | | | | | | | | | | | |
| strate | F | | | | | | | | | | | | |
| with | G | | | ep_{wt} | ep₁ | ep₂ | ep₃ | ep₄ | ep₅ | ep₆ | ep₇ | ep₈ | ep_{wt} |
| product | H | | | | | | | | | | | | |

### Example 3

### Amidase Activity of Various Wildtype Esterases

The amidase activity was determined for various esterases and their activity are shown in percentage relative to the amidase activity of Candida antarctica lipase B. Their relative amino acid sequence identities are also shown relative to that of Candida antarctica lipase B.

TABLE 3
Activity of various wild-type enzymes in amide-bond reaction expressed
as percentage and compared to activity of Candida antarctica lipase B.

| | | Extinction | |
|---|---|---|---|
| | | coefficient used | |
| | Relative | to determine | |
| | amidase | protein | Relative |
| Enzyme | activity | concentration | identity |
| Candida antarctica lipase B | 100% | 1.21 | 100% |
| Hypozyma lipase | 37% | 1.30 | 73% |
| Ustilago maydis lipase | 32% | 1.21 | 68% |
| Aspergillus oryzae caboxypeptidase | 15% | 1.65 | 30% |
| (CP1) | | | |
| Aspergillus oryzae caboxypeptidase | 8% | 1.98 | 29% |
| Y (CPY) | | | |
| Aspergillus oryzae esterase (NN622) | 7% | 1.51 | 29% |
| Bacillus lentus protease (Savinase) | 5% | 0.97 | 29% |
| Bacillus amyloliquefaciens | 0% | 1.08 | 32% |
| protease (BPN′) | | | |

### Example 4

### Amidase Activity of Various Variants

TABLE 4
Activity in amide-bond reaction expressed as improvement factor (IF)
is shown for various lipases. IF was calculated by dividing the activity of
the variant lipase with the activity of its parent lipase. An improvement
factor larger than 1 indicate improved activity in an amide-bond reaction.

| Lipases expressed in | Improvement | Lipases expressed | Improvement |
|---|---|---|---|
| Aspergillus | factor (IF) | in Pichia | factor (IF) |
| Parent lipase | 1.0 | Parent lipase | 1.0 |
| A225K | 1.9-2.2 | L278A | 2.4 |
| A225I | 1.8-3.1 | T103G | 2.9 |
| A225V | 1.2-1.8 | T103V | 1.7 |
| A225L | 1.4 | T103S | 1.4 |
| A225M | 1.1 | T103A | 1.0-1.2 |
| A225S | 1.1 | M129L | 1.4 |
| G39A | 2.6-2.8 | T103G + M129L | 3.2 |
| G39S | 0.6 | D223G | 1.6 |
| G39A + L278A | 5.5 | T42N | 1.3 |
| G39A + W104F | 4.1-4.2 | V190I | 1.0 |
| G39A + Q191R | 1.5 | V190F | 0.2 |
| G39A + L278A + | 1.7 | V190N | 0.2 |
| A281G | | | |
| D223G | 1.5 | V190A | 0.2 |
| D223N | 1.3 | Q157L | 0.2 |
| D223K | 1.0 | Q157A | 0.2 |
| D223R | 0.9 | Q157L + V190F | 0.2 |
| T42G | 1.9 | Q157L + V190I | 0.2 |
| T42S | 1.2 | | |
| Q191R | 1.3 | | |
| I189Y | 1.1 | | |
| I189H | 1.1 | | |
| I189N | 1.0 | | |
| I189Q | 0.4 | | |
| I189D | 0.1 | | |
| I189E | 0.1 | | |
| I189T | 0.1 | | |
| I189S | 0.1 | | |
| T40S | 0.5 | | |
| T40A | 0.1 | | |
| T40G | 0.1 | | |
| W104F | 2.0-2.2 | | |

### Example 5

### Measurement of Activity in Amide-Bond Reaction of Further Samples

After preparation of variants as described in example 1, the following protein samples were concentrated by ultra-filtration and the buffer exchanged to 25 mM phosphate pH 7.0, and measured as described in example 2.

TABLE 5
Activity in amide-bond reaction expressed as improvement factor (IF)
is shown for various samples. IF was calculated by dividing the activity
of the variant lipase with the activity of its parent lipase. An improvement
factor larger than 1 indicate improved activity in an amide-bond reaction.

| Lipases expressed in Aspergillus | Improvement factor (IF) |
|---|---|
| Parent lipase | 1.0 |
| G39A | 1.7 |
| G39A + T103G | 0.8 |
| G39A + W104F | 2.3 |
| G39A + L278A | 3.1-3.3 |
| G39A + T103G + L278A | 2.0-3.8 |
| G39A + W104F + L278A | 5.2-6.3 |
| G39A + T103G + W104F + L278A | 10.0-11.2 |
| G39A + T103G + W104Y + L278A | 2.8 |
| G39A + T103G + W104Q + L278A | 1.9 |
| G39A + W104F + A225K + L278A | 0.8 |
| G39A + W104F + D223N + A225I + L278A | 2.8 |

### Example 6

### Determination of Enzymatic Amide Hydrolysis Activities on Substrate N-benzyl-2-chloroacetamide

Amidase activity of CalB variants was determined in a two-step fluorimetric assay based on the assay described by E. Henke and U. T. Bornscheuer, Anal. Chem. 2003, 75, 255-260. Enzyme preparations after Ni²⁺-affinity chromatography and desalting were used throughout the assays.

First, enzymatic hydrolysis of the amide substrate N-benzyl-2-chloroacetamide was performed in 96-well microtiter plates (Nunc, clear flat-bottom, polystyrene) in 200 uL total volume. All reactions were performed in triplicates on the same microtiter plate. Reaction mixtures contained 20 uL 50 mM amide substrate dissolved in 100% DMSO (final concentrations: 5 mM amide substrate, 10% DMSO), 100 uL 100 mM phosphate buffer pH 7.0 (final concentration: 50 mM), 20 uL 3-30 uM CalB variant (final concentration: 0.3-3 uM), 20 uL 120 ug/mL BSA (final concentration: 12ug/mL), and 40 uL H₂O. To determine the non-enzymatic background amide hydrolysis, identical reactions but without any enzyme were performed in triplicates on each microtiter plate. Microtiter plates were closed with lids and reactions were incubated for 18-20 hours at 37° C. and 220 rpm in a shaker incubator.

In a second step, 50 uL of 20 mM 4-nitro-7-chloro-benzo-2-oxa-1,3-diazole (NBD-CI) dissolved in 1-hexanol was added to the samples, plates were closed with lids, and the reaction of NBD-CI with benzylamine (formed during amide hydrolysis) proceeded for 1 hour at 37° C. and 220 rpm in the same shaker incubator. Fluorescence of the final reaction product was determined using a fluorescence microtiter-plate reader (SpectraMax M3) with excitation at 485 nm and measured emission at 538 nm.

To enable calculation of formed benzylamine during amide hydrolysis reaction, a calibration curve was determined on each microtiter plate with reaction mixtures similar to enzyme hydrolysis samples but without enzyme and with benzylamine instead of the amide substrate N-benzyl-2-chloroacetamide. Calibration curves contained triplicates of samples with a minimum of 8 amine concentrations covering the range between 0.04 mM and 5 mM. Calibration samples were treated as described above for enzyme samples.

For the calculation of enzymatic activities, the average of fluorescence of triplicate measurements was determined and the resulting values of enzyme samples were corrected for the respective values of the background reaction. The specific activity of the assayed CalB variants (v, unit: min⁻¹) was then calculated based on the background-corrected fluorescence values (F_{c}, units: au), the slope m of the linear part of the calibration curve (y=mx+n, with fluorescence values on the y-axis versus benzylamine concentration in mM on the x-axis), the reaction time (t, unit: min), and the enzyme concentration (c_{E}:unit uM) using Formula 1: v=(((F_{c}/m)/t)*1000)/c_{E}

Specific activities v of CalB variants were divided by the respective value for CalB wild-type (also determined using the assay described above) yielding the improvement factors (xᵢ) in amidase activity.

TABLE 6
Specific amidase activities v of CalB variants on substrate N-benzyl-2-
chloroacetamide and resulting improvement factors xᵢ over
CalB wild-type.

| | v | |
|---|---|---|
| Mutation | (min⁻¹) | xᵢ |
| — | 0.022 | 1 |
| G39A + L278A | 0.154 | 7.0 |
| G39A + W104F + L278A | 0.079 | 3.6 |
| G39A + W104F + I189Y + L278A | 0.063 | 2.9 |
| G39A + T103G + W104F + I189Y + L278A | 0.101 | 4.6 |
| G39A + T103G + W104F + D223G + L278A | 0.033 | 1.5 |
| S31R + G39A + T103G + W104F + L278A | 0.163 | 7.4 |
| S31R + G39A + L278A | 0.137 | 6.2 |

### Example 7

### Determination of Enzymatic Amide Hydrolysis Activities on Substrate p-nitrophenyl butyramide

Amidase activity of CalB variants on substrate p-nitrophenyl butyramide (also referred to as “anilide”) was determined in a photometric assay based on the assay described by Syren et al., ChemBioChem 2012, 13, 645-648. Purified enzyme preparations were used throughout the assays.

Enzymatic hydrolysis of the amide substrate p-nitrophenyl butyramide was performed in 96-well microtiter plates (Nunc, clear flat-bottom, polystyrene) in 200 uL total volume. All reactions were performed in triplicates on the same microtiter plate. Reaction mixtures contained 20 uL 10 mM amide substrate dissolved in 100% DMSO (final concentrations: 1 mM amide substrate, 10% DMSO), 100 uL 100 mM phosphate buffer pH 7.0 (final concentration: 50 mM), 20 uL 3-30 uM CalB variant (final concentration: 0.3-3 uM), and 60 uL H₂O. To determine the non-enzymatic background amide hydrolysis, identical reactions but without any enzyme were performed in triplicates on each microtiter plate. Microtiter plates were closed with lids and reactions were incubated for 48 hours at 37° C. and 220 rpm in a shaker incubator. At defined time points during incubation, absorbance at 410 nm of all samples was measured with a microtiter plate SpectraMax M3 spectrophotometer.

For the calculation of enzymatic activities, the average of absorbance of triplicate measurements was calculated (A, unit: au) and reaction slopes (m, unit: au min⁻¹) were determined via linear regression analysis of absorbance values over reaction time (A=mx+n, with absorbance values on the y-axis versus reaction time (t, unit: min) on the x-axis). The resulting reaction slopes m of enzyme samples were corrected for the respective values of the background reaction m_{b} yielding m_{c} (m_{c}=m−m_{b}). The specific activity of the assayed CalB variants (v, unit: min⁻¹) was then calculated based on the background-corrected reaction slopes m_{c}, the extinction coefficient of p-nitroaniline (ε_{410 nm}=9100 M⁻¹ cm⁻¹), the path length (d=0.5 cm), and the enzyme concentration (c_{E}:unit uM) using Formula 1: v=((m_{c}/(ε*d))*1000000)/c_{E}

Specific activities v of CalB variants were divided by the respective value for CalB wild-type (also determined using the assay described above) yielding the improvement factors (xᵢ) in amidase activity (Table 1).

TABLE 7
Specific amidase activities v of CalB variants on substrate p-nitrophenyl
butyramide and resulting improvement factors xᵢ over CalB wild-
type.

| | V | |
|---|---|---|
| Mutation | (min⁻¹) | xᵢ |
| — | 0.0026 | 1 |
| I189Y | 0.0241 | 9.4 |
| I189H | 0.0139 | 5.4 |
| I189A | 0.0128 | 5.0 |
| S31K | 0.0041 | 1.6 |
| I189G | 0.0161 | 6.3 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1-20. (canceled)

21. A variant of a parent lipase which variant is:
a) a polypeptide having at least 60% sequence identity to the mature polypeptide of SEQ ID NO: 2;
b) a polypeptide encoded by a polynucleotide that hybridizes under low stringency conditions with (i) the mature polypeptide coding sequence of SEQ ID NO: 1, or (ii) a sequence encoding the mature polypeptide of SEQ ID NO: 2, or (iii) the full-length complement of (i) or (ii);
c) a polypeptide encoded by a polynucleotide having at least 60% identity to the mature polypeptide coding sequence of SEQ ID NO: 1 or a sequence encoding the mature polypeptide of SEQ ID NO: 2; or
d) a fragment of the mature polypeptide of SEQ ID NO: 2,
wherein said variant has improved activity in an amide-bond reaction in comparison with the parent lipase.

22. The variant of claim 21, comprising a substitution at one or more positions corresponding to positions G39; A225; T103V/S/A; L278G/K/R/H; W104Y/K/R; T42G/S/Q/H/I/L/M; D223G/N/A/V/H; I189; Q191; D134V/T/I/F/A/S/K/R; E188; V221A/R/K/H; 7 P38H/N/A/G; G41P/S; A132; N79; Q106; and/or L140R/K/H of the mature polypeptide of SEQ ID NO: 2.

23. The variant of claim 22, wherein the substitution is G39A; A225I/V/L/M/K/R/S/T/G/H; I189Y/H/Q/N/R/K/A/E/S/T; Q191N/A/R/K/H; E188Q/R/D/N/K/H/A; A132S/N/G/H/K/R; N79V/Q/E; and/or Q106N/Y.

24. The variant of claim 22, further comprising a substitution selected from: T103G; L278A/V; W104F/Q; T42N/V/A; D134L; and G41A.

25. The variant of claim 21, which has at least 85% identity to the amino acid sequence identity to the mature polypeptide of SEQ ID NO: 2.

26. The variant of claim 21, which has at least 90%, sequence identity to the mature polypeptide of SEQ ID NO: 2.

27. The variant of claim 21, which has at least 95% sequence identity to the mature polypeptide of SEQ ID NO: 2.

28. The variant of claim 21, which has at least 98% sequence identity to the mature polypeptide of SEQ ID NO: 2.

29. The variant of claim 21, which has at least 99% sequence identity to the mature polypeptide of SEQ ID NO: 2.

30. The variant of claim 21, wherein the number of substitutions is 1-20, 1-10, 1-5, 1, 2, 3, 4, 5, 6, 7, 8, 9, 10, 11, 12, 13, 14, 15, 16, 17, 18, 19, or 20.

31. The variant of claim 21, which comprises or contains:
a) A225K;
b) A225I;
c) A225V;
d) A225L;
e) A225M;
f) A225S;
g) G39A;
h) D223G;
i) D223N;
j) T42G;
k) T42S;
l) Q191R;
m) I189Y;
n) I189H;
o) T103V;
p) T103S;
q) T103A;
r) G39A+L278A;
s) G39A+W104F;
t) G39A+Q191R;
u) G39A+L278A+A281G;
v) G39A+T103G+L278A;
w) G39A+W104F+L278A;
x) G39A+W104Q+L278A;
y) G39A+T103G+W104F+L278A;
z) G39A+T103G+W104Q+L278A;
aa) G39A+T103G+W104Y+L278A;
bb) G39A+T103G+W104F+D223G+A225K+L278A;
cc) G39A+T103G+W104F+D223N+A225K+L278A;
dd) G39A+W104F+A225K+L278A;
ee) G39A+W104F+D223N+A225K+L278A;
ff) G39A+W104F+D223N+A225I+L278A;
gg) G39A+T103G+D223N+A225K+L278A;
hh) G39A+T103G+W104Y+D223N+A225K+L278A;
ii) G39A+T103G+W104F+M129L+L278A;
jj) G39A+T103G+W104F+M129L+D223N+L278A;
kk) G39A+A225K+L278A;
ll) G39A+D223G+A225K+L278A;
mm) G39A+D223G+A225I+L278A;
nn) G39A+T103G+W104F+A225K+L278A;
oo) G39A+T103G+W104F+A225I+L278A;
pp) G39A+T103G+W104F+D223G+A225I+L278A;
qq) G39A+T103G+W104F+D223N+A225I+L278A;
rr) G39A+W104F+A225I+L278A;
ss) G39A+W104F+V221R+D223N+A225K+L278A;
tt) G39A+T103G+A225K+L278A;
uu) G39A+T103G+A225I+L278A;
vv) G39A+T103G+D223G+A225K+L278A;
ww) G39A+T103G+D223G+A225I+L278A;
xx) G39A+T103G+D223N+A225I+L278A;
yy) G39A+T103G+I189Y+L278A;
zz) G39A+T103G+W104Y+I189Y+D223N+A225K+L278A;
aaa) G39A+W104F+I189Y+D223N+A225K+L278A;
bbb) G39A+T103G+W104F+V221R+D223N+A225K+L278A;
ccc) G39A+T103G+I189Y+D223N+A225K+L278A;
ddd) G39A+T103G+W104Y+I189Y+L278A;
eee) T103G+W104F+D223G+A225K;
fff) T103G+W104F+A225K;
ggg) T103G+W104F+I189Y+D223G;
hhh) T103G+W104F+I189Y;
iii) T103G+W104F+D223G+L278A;
jjj) T103G+W104F+L278A;
kkk) T103G+W104F+Q191R+D223G;
lll) T103G+W104F+Q191R;
mmm) G39A+T42G+T103G+W104F+I189Y+D223G+A225K;
nnn) G39A+T42G+T103G+W104F+D223G+A225K;
ooo) G39A+T42G+T103G+W104F+I189Y+Q191R+D223G+A225K;
ppp) G39A+T42G+T103G+W104F+Q191R+D223G+A225K;
qqq) G39A+W104F+D223G; +A225K
rrr) G39A+W104F+I189Y+D223G; +A225K
sss) G39A+W104F+I189Y; +A225K
ttt) G39A+W104F+A225K;
uuu) G39A+W104F+I189H;
vvv) G39A+W104F+I189Y+D223G;
www) G39A+W104F+I189Y;
xxx) G39A+W104F+D223G; +L278A
yyy) G39A+W104F+I189Y+D223G; +L278A
zzz) G39A+W104F+I189Y; +L278A
aaaa) G39A+W104F+L278A;
bbbb) G39A+T42G+T103G+W104F+M129L+I189Y+D223G+A225K+L278A;
cccc) G39A+T103G+W104F+D223G;
dddd) G39A+T103G+W104F+I189Y+D223G;
eeee) G39A+T103G+W104F+I189Y;
ffff) G39A+T103G+W104F;
gggg) G39A+W104F+I189Y+A281G+A282G+I285A+V286A+L278A;
hhhh) G39A+W104F+A281G+A282G+I285A+V286A+L278A;
iiii) W104F+I189Y+A281G+A282G+I285A+V286A+L278A;
jjjj) W104F+A281G+A282G+I285A+V286A+L278A;
kkkk) G39A+W104F+Q191R;
llll) G39A+W104F+Q191R+D223G;
mmmm) G39A+T42G+T103G+W104F+D223G+A225K+L278A;
nnnn) G39A+T42G+T103G+W104F+D223N+A225K+L278A;
oooo) G39A+T42G+W104F+A225K+L278A;
pppp) G39A+T42G+W104F+D223N+A225K+L278A;
qqqq) G39A+T42G+W104F+D223N+A225I+L278A;
rrrr) G39A+T42G+T103G+D223N+A225K+L278A;
ssss) G39A+T42G+T103G+W104Y+D223N+A225K+L278A;
tttt) T42G+T103G+W104F+D223G+A225K+L278A;
uuuu) T42G+T103G+W104F+D223N+A225K+L278A;
vvvv) T42G+W104F+A225K+L278A;
wwww) T42G+W104F+D223N+A225K+L278A;
xxxx) T42G+W104F+D223N+A225I+L278A;
yyyy) T42G+T103G+D223N+A225K+L278A; or
zzzz) T42G+T103G+W104Y+D223N+A225K+L278A.

32. The variant of claim 21, which has an improved activity in an amide-bond reaction relative to the parent lipase, wherein said reaction is selected from the group consisting of catalytic efficiency, catalytic rate, turnover number, specific activity, substrate binding, substrate cleavage, substrate specificity, substrate stability, and product release.

33. An isolated polynucleotide encoding the variant of claim 21.

34. A nucleic acid construct comprising the polynucleotide of claim 33.

35. An expression vector comprising the polynucleotide of claim 33.

36. A host cell comprising the polynucleotide of claim 33.

37. A method of producing a lipase variant, comprising:
a) cultivating the host cell of claim 36 under conditions suitable for expression of the variant; and
b) recovering the variant.

38. A method of producing a variant of claim 21, comprising:
a) cultivating a transgenic plant or a plant cell comprising a polynucleotide encoding the variant under conditions conducive for production of the variant; and
b) recovering the variant.

39. A method for obtaining a variant from a parent lipase which variant as compared to the parent lipase has improved activity in amide-bond reaction comprising:
a) selecting at least one amino acid residue located within 10 Å of any of the catalytic triade residues which (i) changes the geometry of the pocket that contains the catalytic triade; and/or (ii) affects the catalytic triade through the electrostatic field;
b) making a substitution of the at least one amino acid residue;
c) preparing the variant resulting from step (a) & (b);
d) testing the activity of the variant in an amide-bond reaction; and
e) selecting the variant which compared to the parent lipase has improved activity in an amide-bond reaction.

40. The method of claim 39, wherein the substitution at the at least one amino acid residue is selected from: G39; A225; T103V/S/A; L278G/K/R/H; W104Y/K/R; T42G/S/Q/H/I/L/M; D223G/N/A/V/H; I189; Q191; D134V/T/I/F/A/S/K/R; E188; V221A/R/K/H; P38H/N/A/G; G41P/S; A132; N79; Q106; and/or L140R/K/H corresponding to positions in the mature polypeptide of SEQ ID NO: 2.


SEQUENCE LISTING (6 sequences)

SEQ ID NO: 1 - DNA, 1029 bp, Candida antarctica
  CDS (1)..(1026)
  sig_peptide (1)..(75)
  mat_peptide (76)..(1026)
atgaagctactctctctgaccggtgtggctggtgtgcttgcgacttgcgttgcagccactcctttggtgaagcgtctaccttccggttcggaccctgccttttcgcagcccaagtcggtgctcgatgcgggtctgacctgccagggtgcttcgccatcctcggtctccaaacccatccttctcgtccccggaaccggcaccacaggtccacagtcgttcgactcgaactggatccccctctcaacgcagttgggttacacaccctgctggatctcacccccgccgttcatgctcaacgacacccaggtcaacacggagtacatggtcaacgccatcaccgcgctctacgctggttcgggcaacaacaagcttcccgtgcttacctggtcccagggtggtctggttgcacagtggggtctgaccttcttccccagtatcaggtccaaggtcgatcgacttatggcctttgcgcccgactacaagggcaccgtcctcgccggccctctcgatgcactcgcggttagtgcaccctccgtatggcagcaaaccaccggttcggcactcaccaccgcactccgaaacgcaggtggtctgacccagatcgtgcccaccaccaacctctactcggcgaccgacgagatcgttcagcctcaggtgtccaactcgccactcgactcatcctacctcttcaacggaaagaacgtccaggcacaggccgtgtgtgggccgctgttcgtcatcgaccatgcaggctcgctcacctcgcagttctcctacgtcgtcggtcgatccgccctgcgctccaccacgggccaggctcgtagtgcagactatggcattacggactgcaaccctcttcccgccaatgatctgactcccgagcaaaaggtcgccgcggctgcgctcctggcgccggcagctgcagccatcgtggcgggtccaaagcagaactgcgagcccgacctcatgccctacgcccgcccctttgcagtaggcaaaaggacctgctccggcatcgtcaccccctga

SEQ ID NO: 2 - protein, 342 aa, Candida antarctica
MKLLSLTGVAGVLATCVAATPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 3 - protein, 319 aa, Hyphozyma sp.
FTPFPTGADPAFTQSQATLDAGLTCQSGSPSSQKNPILLVPGTGNTGPQSFDSNWIPLSAQLGYSPCWVSPPPFMLNDSQINAEYIVNAIHTLSSGSGSKVPVLTWSQGGLAAQWALTFFPSTRNKVDRLMAFAPDYKGTVEAGLLDAFGLSAPSVWQQTAQSAFVTALDQAGGLNQIVPTTNLYSATDEVVQPQFANGPPDSSYLSNGKNIQAQSICGPLFIIGHAGSLYSQFSYVVGKSALASPTGQAQSSDYSIKDCNPAPANPLTAQQKLDSAAIILVAGKNIVTGPKQNCEPDLMPYARKYRIGKKTCSGVITG

SEQ ID NO: 4 - protein, 336 aa, Ustilago maydis
MKTTSVISALVTLASIIRAAPLASSDPAFSTPKATLDAGLECQTGSPSSQTKPILLVPGTGANGTQTFDSSWIPLSAKLGFSPCWISPPPFMLNDSQVNVEYIVNAVQTLYAGSGSKKVPVLTWSQGGLATQWALTFFPSIRNQVDRLMAFAPDYKGTIEAGLLSTFGLASQSVWQQQAGSAFVTALKNAGGLTSFVPTTNLYSFFDEIVQPQVFNSDADSSYLGNSKNIQAQTVCGGFFVIDHAGSLTSQFSYVVGKSALTSSSGVANSADYSSKDCKASPADDLSAKQKADASALLFVAAGNLLAGPKQNCEPDLKPYARQFAVGKKTCSGTIN

SEQ ID NO: 5 - protein, 341 aa, Sporisorium reilianum
MKFLTALTVLASCSALASATPLVKRLPSGSDPAYTLSKAQLDSVLACQNGSPSSQKNPILLVPGTGTTGPQSFDSNWIPLSTQLGYSPCWVSPPPFMLNDTQVNAEYIVNAVKVLSSASGAKVPVLTWSQGGLAAQWALTFFPSIRTQVDRLMAFAPDYKGTVLAAFLTTPGLASESVWQQQAGSALTTALANAGGLTKIVPTTNLYSATDDIVQPQTFNGPLDSGYLNGGAKNIQAQSVCGPLFVVDHAGTLTSQFSFVVGRSALRSTTGQAQSKDYGVTDCNPLPADSLTPDQKLRAEGLLLVAGANVAAGPKQNCEPDLMPYARQYAVGKRTCSGVIL

SEQ ID NO: 6 - protein, 316 aa, Cryptococcus tsukubaensis
LPSGSDPAFSTPQSTLAAGLTCQSGSPSSVSNPILLVPGTGTTGPQSFDSNWIPLSTMLGYTPCWVSPPPYMLNDSQINAEYIVNAVNTLYSGSGSKKVPVLTWSQGGLATQWALTFFPSIRSKVDRLMAFAPDYKGTIEAGLLDAVGASAPSVWQQTAGSAFVTALQNAGGLNEIIPTTNLYSATDDIVQPQVTNSPLDSSYLFNGKNIQAQSVCGPLFIIDHAGSLTSQFSYVVGKSALVSLTGKAQSSDYGLKDCNPLPADNLTAQQKLQAEGLLLVAGANLATGPKQNCEPDLMPYARRYAFGKKTCSGIIL